(19) United States
(12) Patent Application Publication
(10) Pub. No.: US 2019/0049535 A1
(43) Pub. Date: Feb. 14, 2019

(54) SYSTEM AND METHOD FOR ELIMINATING SHIELD CURRENT FROM RADIO FREQUENCY (RF) BODY COIL CABLES IN A MAGNETIC RESONANCE IMAGING (MRI) SYSTEM

(71) Applicant: General Electric Company, Schenectady, NY (US)

(72) Inventor: Saikat Saha, Pewaukee, WI (US)

(21) Appl. No.: 15/676,073

(22) Filed: Aug. 14, 2017

(51) Int. Cl. G01R 33/385; G01R 33/34; A61B 5/055; G01R 33/422

(52) U.S. Cl. CPC G01R 33/385; G01R 33/34023; G01R 33/422; G01R 33/34046; A61B 5/055

(57) ABSTRACT

A (MRI) system includes a RF coil assembly, a gradient coil assembly disposed around the RF coil assembly, the gradient coil assembly including a RF shield, and a superconducting magnet assembly disposed around the gradient coil assembly. The superconducting magnet assembly including a vessel containing a plurality of superconducting coils. The RF coil assembly including a plurality of RF coil elements applied on an outer surface of a hollow cylindrical RF coil former. The plurality of RF coil elements including a pair of end rings with a plurality of rungs positioned between the pair of end rings, a plurality of ground patches, and a plurality of RF cables. A shield of each of the plurality of RF cables is attached to a ground patch of the plurality of ground patches and each ground patch is capacitively coupled to the RF shield.

### BACKGROUND

Embodiments of the subject matter disclosed herein relate to magnetic resonance imaging (MRI) systems and methods, and more particularly, to a system and method for eliminating shield current from radio frequency (RF) body coil cables in a MRI system.

Magnetic resonance imaging (MRI) systems provide a widely accepted and commercially available technique for obtaining digitized visual images representing the internal structure and tissue of subjects having atomic nuclei that are susceptible to nuclear magnetic resonance (NMR). MRI systems include a superconducting magnet assembly that creates a strong, uniform, static, polarizing magnetic field designated as B₀, which is imposed on the nuclei of the subjects being imaged. When a subject is placed in the polarizing magnetic field B₀, the nuclear spins associated with the nuclei in tissue, fluid or other structures become polarized, wherein the individual magnetic moments associated with these spins attempts to align with the polarizing magnetic field B₀, but process about it in random order at their characteristic Larmor frequency, resulting in a small net tissue magnetization along that axis.

MRI systems also include gradient coils that produce smaller amplitude, spatially-varying gradient magnetic fields with orthogonal axes to spatially encode a MR signal by creating a signature resonance frequency at each location in the subject's body. The MRI systems also include RF coils. The RF coils are used to transmit RF excitation signals at or near the resonance frequency of the nuclei, also referred to as the Larmor frequency, which add energy to the nuclear spins of the nuclei. As the nuclear spins relax back to their lower energy normal state, the nuclei release the absorbed energy in the form of a RF signal. This RF signal is detected by the MRI system (RF coil) and is transformed into images of the internal structures and tissue of the subject being imaged.

Various types of RF coils may be used in an MRI system such as a whole-body RF coils (RF body coils) and RF surface (or local) coils. A common RF body coil configuration is the birdcage coil. The RF body coil includes RF cables between a RF transmitter and RF amplifier to RF coil elements, and then from the RF coil elements to a pre-amplifier and RF receiver.

A RF cable which is in proximity of high electromagnetic field regions, may carry a significant amount of unwanted current on the outside of the RF cable shield. The presence of several such RF cables near each other introduces spurious RF interactions. To minimize unwanted current on the outside of a RF cable shield, baluns are typically included on the RF cables. The baluns decouple the RF cable shields from induced current on the outside of the RF cable shield. However, baluns are difficult to implement because they are big and bulky, their use often constrained by space availability, often need precise tuning to be effective, and are expensive.

Other techniques to minimize unwanted current on the outside of a RF cable shield include implementing a quarter wave sleeve and/or employing stub baluns on the RF cables. However, such techniques require a much wider patient bore tube, which can create other significant challenges such as the redesign of the MRI system's resonance assembly.

Accordingly, there is a need for an improved and simplified system and method for eliminating unwanted current on the outside of RF cable shields without the need for additional shielding, use of baluns, and/or redesign of the resonance assembly.

### SUMMARY

In accordance with an aspect, a resonance assembly of a magnetic resonance imaging (MRI) system includes a superconducting magnet assembly, a gradient coil assembly disposed within an inner diameter of the superconducting magnet, the gradient coil assembly including a RF shield, and a RF coil assembly disposed within an inner diameter of the gradient coil assembly. The RF coil assembly including a pair of end rings with a plurality of rungs positioned between the pair of end rings, a plurality of ground patches spaced apart from at least one of the pair of end rings, and a plurality of RF cables, each RF cable having a plurality of conductors and a shield positioned around the plurality of conductors in a coaxial cable configuration. The shield of each of the plurality of RF cables is attached to a ground patch of the plurality of ground patches. Each of the plurality of ground patches are capacitively coupled to the RF shield.

In accordance with another aspect, a magnetic resonance imaging (MRI) system includes a RF coil assembly, a gradient coil assembly disposed around the RF coil assembly, the gradient coil assembly including a RF shield, and a superconducting magnet assembly disposed around the gradient coil assembly. The superconducting magnet assembly including a vessel containing a plurality of superconducting coils. The RF coil assembly includes a pair of end rings with a plurality of rungs positioned between the pair of end rings, a plurality of ground patches spaced apart from at least one of the pair of end rings, and a plurality of RF cables, each RF cable having a plurality of conductors and a shield positioned around the plurality of conductors in a coaxial cable configuration. The shield of each of the plurality of RF cables is attached to a ground patch of the plurality of ground patches. The plurality of ground patches are capacitively coupled to the RF shield.

In accordance with yet another aspect, a method of eliminating the shield current in an RF cable of a MRI system including providing a plurality of ground patches, each of the plurality of ground patches electrically coupled to a RF cable shield of a plurality of RF cables and each of the plurality of ground patches capacitively coupled to a RF shield. The method further including applying the plurality of ground patches on an outer cylindrical surface of a RF coil former. The plurality of ground patches are spaced apart from the RF shield with a dielectric material in between.

Various other features and advantages will be made apparent from the following detailed description and the drawings.

### BRIEF DESCRIPTION OF THE DRAWINGS

FIG. 1 is a schematic block diagram of an exemplary magnetic resonance imaging (MRI) system in accordance with an embodiment;

FIG. 2 is a cross-sectional diagram of an exemplary resonance assembly of the MRI system of FIG. 1;

FIG. 3 is a perspective diagram of an exemplary RF coil assembly in accordance with an embodiment;

FIG. 4 is an enlarged schematic diagram of a single end ring and a single ground patch with a RF cable shield of a RF cable electrically connected to the ground patch of the exemplary RF coil assembly of FIG. 3;

FIG. 5 is a cross-sectional diagram of a portion of an exemplary resonance assembly in accordance with an embodiment; and

FIG. 6 is an enlarged schematic diagram of a portion of the exemplary resonance assembly of FIG. 5.

### DETAILED DESCRIPTION

Eliminating unwanted current on the outside of radio frequency (RF) cable shields of a RF coil assembly in a MRI system is very important. However, the physical and structural complexities and constraints of the RF coil assembly, gradient coil assembly and RF shield make it difficult to eliminate the unwanted current. Embodiments of the subject matter disclosed herein provide a RF body coil for a MRI system utilizing ground patches of electrically conductive material where the shield of each RF cable is attached to a ground patch and each ground patch is capacitively coupled to the RF shield, creating an improved, cost effective, and reliable system and method for eliminating unwanted current on the outside of RF cable shields in a MRI system, without the need for additional shielding, use of baluns, and/or redesign of the resonance assembly.

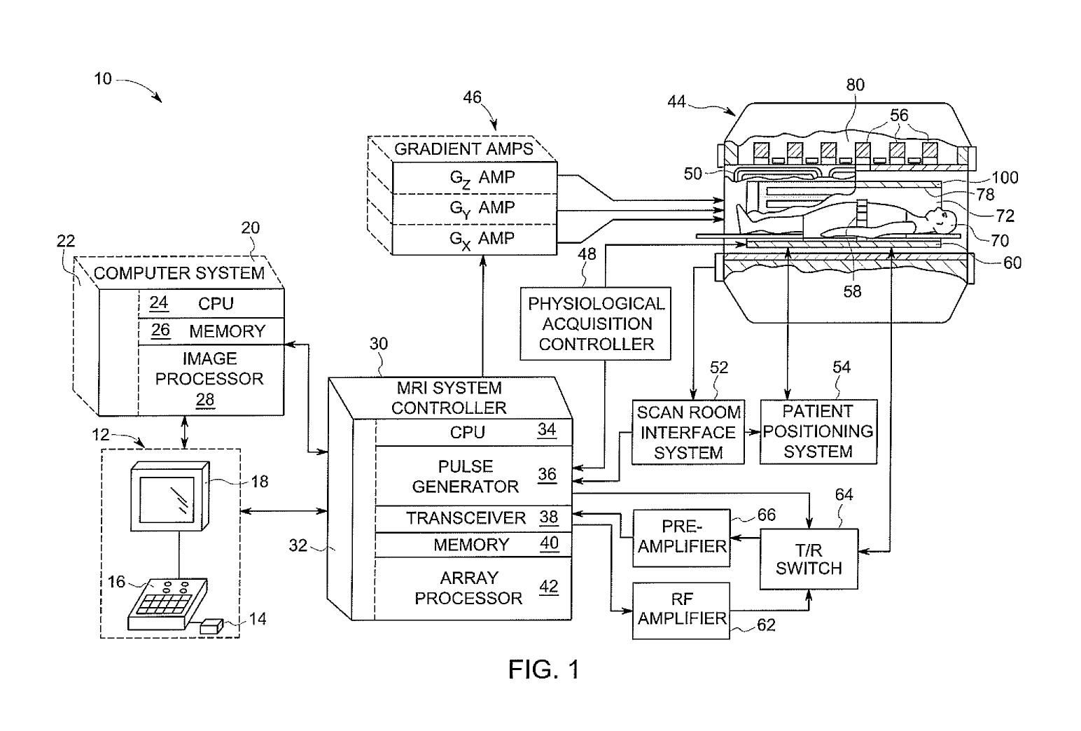

FIG. 1 is a schematic block diagram of an exemplary magnetic resonance imaging (MRI) system 10 in accordance with an embodiment. The operation of MRI system 10 is controlled from an operator workstation 12 that includes an input device 14, a control panel 16, and a display 18. The input device 14 may be a joystick, keyboard, mouse, track ball, touch activated screen, voice control, or any similar or equivalent input device. The control panel 16 may include a keyboard, touch activated screen, voice control, buttons, sliders, or any similar or equivalent control device. The operator workstation 12 is coupled to and communicates with a computer system 20 that enables an operator to control the production and viewing of images on display 18. The computer system 20 includes a plurality of modules that communicate with each other via electrical and/or data connections 22. The computer system connections 22 may be direct wired connections, fiber optic connections, wireless communication links, or the like. The modules of the computer system 20 include a central processing unit (CPU) module 24, a memory module 26, which may include a frame buffer for storing image data, and an image processor module 28. In an alternative embodiment, the image processor module 28 may be replaced by image processing functionality in the CPU module 24. The computer system 20 may be connected to archival media devices, permanent or back-up memory storage, or a network. The computer system 20 is coupled to and communicates with a separate MRI system controller 30.

The MRI system controller 30 includes a plurality of modules in communication with each other via electrical and/or data connections 32. The MRI system controller connections 32 may be direct wired connections, fiber optic connections, wireless communication links, or the like. The modules of the MRI system controller 30 include a CPU module 34, a pulse generator module 36, a transceiver 38, a memory module 40, and an array processor 42. In an alternative embodiment, the pulse generator module 36 may be integrated into the resonance assembly 44 of the MRI system. The MRI system controller 30 is coupled to and receives commands from the operator workstation 12 to indicate the MRI scan sequence to be performed during a MRI scan. The MRI system controller 30 is also coupled to and communicates with a gradient amplifier system 46, which is coupled to a gradient coil assembly 50 to produce magnetic field gradients during a MRI scan.

The pulse generator module 36 may also receive data from a physiological acquisition controller 48 that receives signals from a plurality of different sensors connected to a patient or subject 70 undergoing a MRI scan, such as electrocardiography (ECG) signals from electrodes attached to the patient. And the pulse generator module 36 is coupled to and communicates with a scan room interface system 52, which receives signals from various sensors associated with the condition of the resonance assembly 44. The scan room interface system 52 is also coupled to and communicates with a patient positioning system 54, which sends and receives signals to control movement of a patient table to a desired position for a MRI scan.

The MRI system controller 30 provides gradient waveforms to the gradient amplifier system 46, which is comprised of Gx, Gy and Gz gradient amplifiers. Each Gx, Gy and Gz gradient amplifier excites a corresponding gradient coil in the gradient coil assembly 50 to produce magnetic field gradients used for spatially encoding MR signals during a MRI scan. The gradient coil assembly 50 is included within the resonance assembly 44, which also includes a superconducting magnet assembly 80 having superconducting coils 56, which in operation, provides a homogenous longitudinal main magnetic field B₀ within an open cylindrical imaging volume 72 that is enclosed by resonance assembly 44. The resonance assembly 44 also includes a RF coil assembly 100, which includes a RF body coil 60, which in operation, provides a uniform RF magnetic field Bi that is generally perpendicular to B₀ throughout the open cylindrical imaging volume 72. The resonance assembly 44 may also include RF surface coils 58 used for imaging different anatomies of a patient 70 undergoing a MRI scan. The RF body coil 60 and RF surface coils 58 may be configured to operate in a transmit and receive mode, transmit mode, or receive mode.

A patient or subject 70 undergoing a MRI scan may be positioned within the open cylindrical imaging volume 72 of the resonance assembly 44. The transceiver module 38 in the MRI system controller 30 produces RF excitation pulses that are amplified by a RF amplifier 62 and provided to the RF body coil 60 and RF surface coils 58 through a transmit/receive switch (T/R switch) 64.

As mentioned above, RF body coil 60 and RF surface coils 58 may be used to transmit RF excitation pulses and/or to receive resulting MR signals from a patient undergoing a MRI scan. The resulting MR signals emitted by excited nuclei in the patient undergoing a MRI scan may be sensed and received by the RF body coil 60 or RF surface coils 58 and sent back through the T/R switch 64 to a pre-amplifier 66. The amplified MR signals are demodulated, filtered and digitized in the receiver section of the transceiver 38. The T/R switch 64 is controlled by a signal from the pulse generator module 36 to electrically connect the RF amplifier 62 to the RF body coil 60 during the transmit mode and connect the pre-amplifier 66 to the RF body coil 60 during the receive mode. The T/R switch 64 may also enable RF surface coils 58 to be used in either the transmit mode or receive mode.

The resulting MR signals sensed and received by the RF body coil 60 or RF surface coils 58 are digitized by the transceiver module 38 and transferred to the memory module 40 in the MRI system controller 30.

A MR scan is complete when an array of raw k-space data, corresponding to the received MR signals, has been acquired and stored temporarily in the memory module 40 until the data is subsequently transformed to create images. This raw k-space data is rearranged into separate k-space data arrays for each image to be reconstructed, and each of these separate k-space data arrays is input to the array processor 42, which operates to Fourier transform the data into arrays of image data.

The array processor 42 uses a known transformation method, most commonly a Fourier transform, to create images from the received MR signals. These images are communicated to the computer system 20 where they are stored in memory module 26. In response to commands received from the operator workstation 12, the image data may be archived in long-term storage or it may be further processed by the image processor module 28 and conveyed to the operator workstation 12 for presentation on the display 18.

In alternative embodiments, the modules of computer system 20 and MRI system controller 30 may be implemented on the same computer system or a plurality of computer systems.

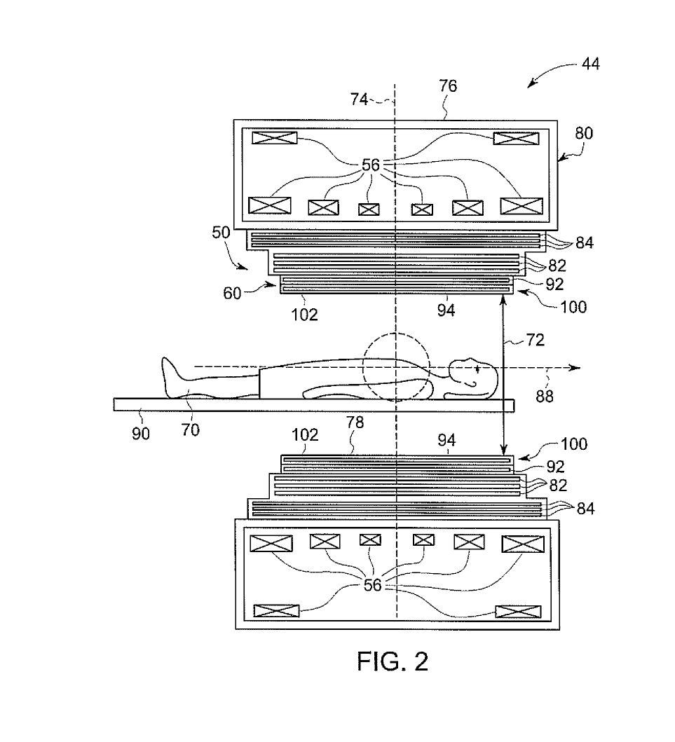

FIG. 2 is a cross-sectional diagram of an exemplary resonance assembly 44 of the MRI system 10 of FIG. 1. Resonance assembly 44 is cylindrical in shape having a vertical center axis 74, a longitudinal center axis 88, and includes, among other elements, a superconducting magnet assembly 80, a gradient coil assembly 50 and a RF coil assembly 100. Various other elements such as covers, supports, suspension members, end caps, brackets, etc. are omitted from FIG. 2 for clarity.

An open cylindrical imaging volume 72 is surrounded by a patient bore tube 78. RF coil assembly 100 is cylindrical and is disposed around the patient bore tube 78 and mounted inside of and adjacent to cylindrical gradient coil assembly 50. The gradient coil assembly 50 is disposed around the RF coil assembly 100 in a spaced-apart coaxial relationship and the gradient coil assembly 50 circumferentially surrounds the RF coil assembly 100. Gradient coil assembly 50 is mounted inside of and adjacent to superconducting magnet assembly 80 and is circumferentially surrounded by superconducting magnet assembly 80.

As shown in FIG. 2, resonance assembly 44 includes a superconducting magnet assembly 80, a gradient coil assembly 50 disposed within an inner diameter of the superconducting magnet assembly 80, the gradient coil assembly 50 including a RF shield 92, and a RF coil assembly 100 disposed within an inner diameter of the gradient coil assembly 50.

A patient or subject 70 undergoing a MRI scan may be placed into the resonance assembly 44 along a longitudinal center axis 88 (e.g., z axis) on a patient table 90. Center axis 88 is centered within open cylindrical imaging volume 72 of resonance assembly 44 and along the direction of the main magnetic field, B₀, generated by superconducting magnet assembly 80. RF body coil 60 may be used to apply RF excitation pulses to the patient 70 and may be used to receive emitted RF signals back from the patient. Gradient coil assembly 50 generates magnetic field gradients used for spatially encoding MR signals during a MRI scan.

Superconducting magnet assembly 80 may include, for example, several radially aligned and longitudinally spaced apart superconductive coils 56, each capable of carrying a large current. The superconductive coils 56 are designed to create a homogenous longitudinal magnetic field, B₀, within the open cylindrical imaging volume 72. The superconductive coils 56 are enclosed in a vessel 76. The vessel 76 is designed to maintain the temperature of the superconducting coils 56 below the appropriate critical temperature so that the superconducting coils 56 are in a superconducting state with zero resistance. Vessel 76 may include, for example, a helium vessel (not shown) and thermal shields (not shown) for containing and cooling the superconducting coils in a known manner. The vessel 76 is configured to maintain a vacuum and to prevent heat from being transferred to the superconducting magnet assembly 80.

Gradient coil assembly 50 comprises a cylindrical inner gradient coil set 82 and a cylindrical outer gradient coil set 84 positioned in concentric arrangement with respect to the longitudinal center axis 88. Inner gradient coil set 82 includes X, Y and Z gradient coils and outer gradient coil set 84 includes the respective outer X, Y and Z gradient coils. The gradient coils may be activated by passing an electric current through the coils to generate a gradient field in the open cylindrical imaging volume 72 as required in MR imaging. The gradient coil assembly 50 also includes a cylindrical RF shield 92 that is integrated within the gradient coil assembly 50 and is adjacent to and formed about an inner portion of the inner gradient coil set 82.

The RF shield 92 is the innermost layer of the gradient coil assembly 50. The RF shield 92 may be made of an electrically conductive material, such as copper, a stainless steel mesh, or any other electrically conductive material and may be potted with an epoxy resin or a glass tape.

The RF shield 92 functions as an essential element of the MRI system 10, providing a ground or current return path for RF coil elements 102 of the RF body coil 60, and to shield RF signals and electromagnetic interference from the gradient coil assembly 50 and superconducting magnet assembly 80. The RF shield 92 isolates the RF body coil 60 from the gradient coil assembly 50.

The resonance assembly 44 also includes a RF coil assembly 100, which includes a RF coil former 94 and RF coil elements 102 to create RF body coil 60. The RF coil assembly 100 may be mounted inside the gradient coil assembly 50 in a spaced apart coaxial relationship. The RF coil assembly 100 functions to provide a uniform RF magnetic field Bi that is generally perpendicular to B₀ throughout the open cylindrical imaging volume 72. The RF body coil 60 also transmits RF excitation pulses and receives resulting MR signals from a patient undergoing a MRI scan.

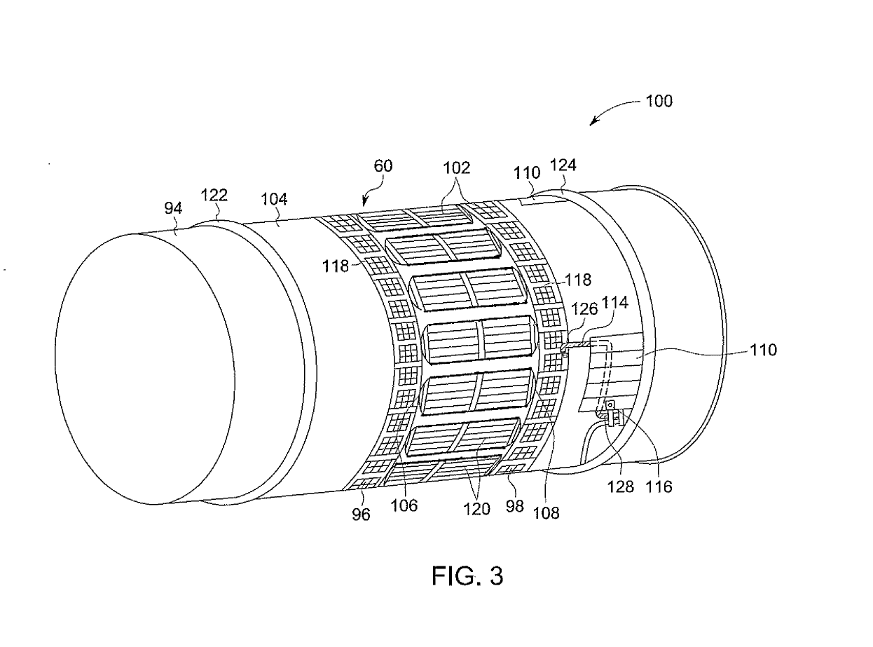

FIG. 3 is a perspective diagram of an exemplary RF coil assembly 100 in accordance with an embodiment. The RF coil assembly 100 includes a RF coil former or hollow cylindrical tube 94 and a plurality of RF coil elements 102 mounted around an outer cylindrical surface 104 of the RF coil former 94 to create a RF body coil 60. The RF coil former 94 may be composed of a fiberglass or fiber reinforced plastic (FRP) cylinder, although it is recognized that other suitable materials may also be used.

Various types of RF body coils may be used in an MRI system such as a whole-body RF coil (RF body coil) and RF surface (or local) coils. A common RF body coil configuration is the birdcage coil. The RF coil assembly 100 shown in FIG. 3 illustrates a RF body coil having a birdcage configuration. In other embodiments of the subject matter disclosed herein, the RF body coil may also be implemented as a transverse electromagnetic (TEM) RF body coil, micro-strip loop RF body coil, and other types of RF body coils configurations.

The RF coil elements 102 include electrically conductive elements that form a magnetically inductive structure that can generate and/or receive RF magnetic fields polarized in a plane transverse (perpendicular) to the direction of the main static polarizing magnetic field B₀. Capacitive elements (not shown) may be added to the inductive elements to make the RF body coil resonant to a specific narrow band of frequencies centered around the Larmor frequency of the nucleus of interest at the specific static polarizing magnetic field strength. This resonant RF body coil is impedance matched to RF cables with an RF network. In many cases, decoupling networks or circuitry (not shown) is employed to disable the RF body coil resonance during RF transmit or RF receive modes.

The RF coil elements 102 include a pair of end rings 96, 98 and a plurality of rungs 120 extending between the end rings 96, 98. The pair of end rings 96, 98 include a first end ring 96 and a second end ring 98 that are disposed around the outer cylindrical surface 104 of the RF coil former 94 towards the ends 122, 124 of the RF coil former 94, generally at what is referred to as the “patient end” and the “service end” of the RF coil assembly 100. Each of the end rings 96, 98 may be composed of a plurality of segments 118 and may include capacitors (not shown) positioned between the segments 118. Decoupling networks or circuitry (not shown) may also be positioned on end rings 96, 98 to decouple the RF body coil 60 from other coils in the resonance assembly 44.

In an exemplary embodiment, the first 96 and second 98 end rings may be constructed from conventional materials with high electrical conductivity such as copper or other electrically conductive materials. In various embodiments, the first 96 and second 98 end rings may be a conductive foil, a conductive tape, thin sheets of electrically conductive material, etc. that are attached around the outer cylindrical surface 104 of the RF coil former 94.

The plurality of rungs 120 extend longitudinally on the outer cylindrical surface 104 of the RF coil former 94 between the first 96 and second 98 end rings. Each rung 120 has a first end 106 and a second end 108 that is opposite the first end. The first end 106 of each rung 120 is electrically coupled to the first end ring 96 and the second end 108 of each rung 120 is electrically coupled to the second end ring 98. The RF coil assembly 100 also includes a plurality of RF cables 114 coupled to the RF coil elements 102.

An exemplary number of rungs 120 are shown in FIG. 3. However, fewer or more rungs may be used depending upon specific design requirements. The plurality of rungs 120 are arranged cylindrically around the outer cylindrical surface 104 of the RF coil former 94 and may be, for example, uniformly spaced apart from one another. The plurality of rungs 120 may be constructed from conventional materials with high electrical conductivity such as copper or other electrically conductive materials. In various embodiments, the plurality of rungs 120 may be a conductive foil, a conductive tape, thin sheets of electrically conductive material, etc. that are attached around the outer cylindrical surface 104 of the RF coil former 94.

The plurality of RF cables 114 are routed along the RF coil former 94 and are electrically coupled to RF coil elements 102. The plurality of RF cables 114 may be routed along a portion of the outer cylindrical surface 104 and a portion of an inner cylindrical surface (not shown) of the RF coil former 94. The plurality of RF cables 114 allow connection between a RF transmitter and RF amplifier to RF coil elements 102, and from the RF coil elements 102 to a pre-amplifier and RF receiver on the MRI system.

The number of RF cables employed to power and drive the RF body coil may vary. However, RF cables are provided in multiples of two (e.g., 2, 4, 6, 8, etc.). In one example, a RF body coil having sixteen rungs, four RF cables may be used to drive the RF body coil in quadrature (i.e., RF cables being 90 degrees apart in phase). In other examples, a RF body coil having sixteen rungs, eight RF cables may be used to drive the RF body coil with the RF cables being 45 degrees apart in phase, or twelve RF cables may be used to drive the RF body coil with the RF cables being 30 degrees apart in phase.

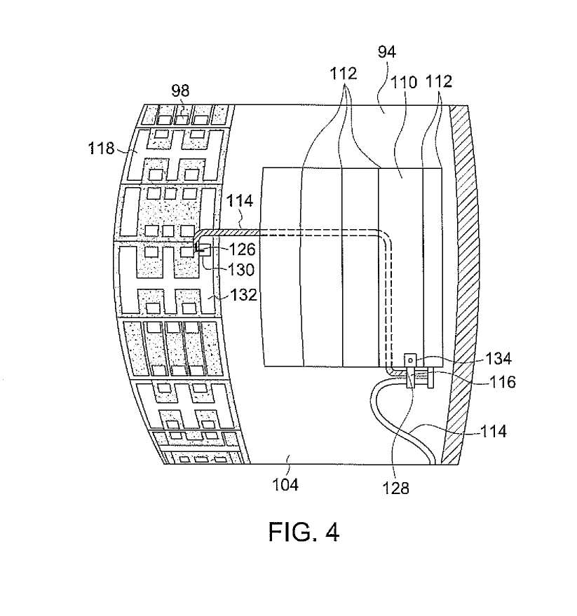

In RF antenna such as RF body coils, any cable, which is in the proximity of electromagnetic fields, carry unwanted current on the outside of the RF cable shield. To eliminate such shield currents on RF cables, baluns are typically used. Sometimes baluns are difficult to implement because they are big and bulky, need precise tuning for them to be effective, and are expensive. Therefore, a plurality of ground patches 110 are electrically coupled to the shields 116 of the RF cables 114 as shown in FIG. 4.

A plurality of ground patches 110 are disposed around the outer cylindrical surface 104 of RF coil former 94 outside of at least one of the end rings 98 near an end 124 of RF coil former 94. The ground patches 110 remove unwanted current flowing on the outside of the RF cable shield 116. A first end 126 of the RF cable 114 is connected to the end ring 98 of the RF body coil 60 and the second end 128 of RF cable 114 is connected to a RF amplifier passing through the ground patch 110. The shield 116 of RF cable 114 is electrically connected to ground patch 110 to eliminate unwanted shield current from the RF cable shield 116.

In an exemplary embodiment, the number of ground patches should be the same as the number of RF cables, such that the shield from each RF cable is electrically coupled to a single ground patch. In one example, if four RF cables are used, then there will be four ground patches employed, one for each RF cable.

In an exemplary embodiment, the plurality of ground patches 110 may be constructed from conventional materials with high electrical conductivity such as copper or other electrically conductive materials. In various embodiments, the plurality of ground patches 110 may be a conductive foil, a conductive tape, thin sheets of electrically conductive material, etc. that are attached around the outer cylindrical surface 104 of the RF coil former 94.

FIG. 4 is an enlarged schematic diagram of second end ring 98 and a ground patch 110 with a RF cable shield 116 of a RF cable 114 electrically connected to the ground patch 110 of the exemplary RF coil assembly 100 of FIG. 3. The plurality of rungs 120 and the first end ring 96 are not shown in FIG. 4. The RF coil former 94 composed of a fiberglass or FRP cylinder acts as a substrate for the second end ring 98 and ground patch 110 that are applied on its outer cylindrical surface 104. The plurality of rungs 120, first 96 and second 98 end rings, and plurality of ground patches 110 may be potted with an epoxy resin or a glass tape.

In the plurality of RF cables 116, each RF cable 116 having a plurality of conductors and a shield positioned around the plurality of conductors in a coaxial cable configuration. A first end 126 of each of the plurality of RF cables 114 is electrically connected to at least one end ring 98 at a desired point 130 of end ring segments 118, along an outer edge 132 of end ring 98 (i.e., on a side opposite or distal from an edge coupled to the rungs), such as by soldering or other electrical bonding method. Each of the plurality of RF cables 114 pass under the ground patch 110 for electrical connection of the RF cable shield 116 to the ground patch 110 at a desired point 134, such as by soldering or other electrical bonding method. A second end 128 of each of the plurality of RF cables 114 is to be connected to the T/R switch 64 of the MRI system. In an exemplary embodiment, each RF cable shield 116 of each of the plurality of RF cables 114 is electrically connected to a single ground patch 110. This connection eliminates electromagnetic field induced currents on the outer shield of the RF cables.

Each ground patch 110 of the plurality of ground patches is spaced apart from one of the end rings 98 to minimize coupling. Each ground patch 110 preferably includes a plurality of slits 112 at various locations along its length or width to minimize eddy current generation from the gradient coils. The dimensions of the ground patches 110 are important so that they have very low impedance at the frequency of interest and couple well with the RF shield 92. Each ground patch 110 functions as a balun to eliminate unwanted shield current from each of the plurality of RF cables 114. Eliminating the use of baluns on the RF cables reduces cost and improves reliability of the RF coil assembly.

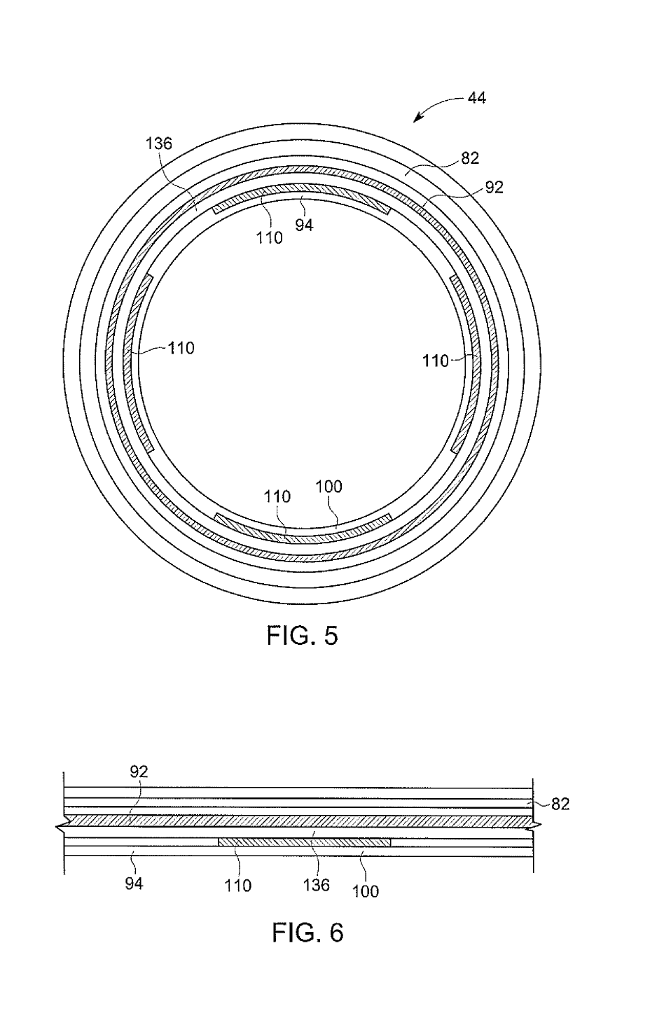

FIG. 5 is a cross-sectional diagram of a portion of an exemplary resonance assembly 44 in accordance with an embodiment. The superconducting magnet assembly 80 and outer gradient coil set 84 of the gradient coil assembly are not shown in FIG. 5. As shown in FIG. 5, the RF coil assembly 100 is mounted inside of the inner gradient coil set 82 in a spaced apart coaxial relationship. The RF coil assembly 100 includes a plurality of ground patches 110 applied on an outer cylindrical surface 104 of a RF coil former 94.

A RF shield 92 integrated within and positioned adjacent to the inner gradient coil set 82 is spaced apart from the plurality of ground patches 98 by a dielectric material 136. The RF shield 92 is the innermost layer of inner gradient coil set 82 and located closest to the ground patches 110. The plurality of ground patches 110 are spaced apart from the RF shield 92 with a dielectric material 136 in between the plurality of ground patches 110 and the RF shield 92. Each ground patch 110 capacitively couples to the RF shield 92. There is no physical connection between the plurality of ground patches 110 and the RF shield 92.

FIG. 6 is an enlarged schematic diagram of a portion of the exemplary resonance assembly 44 of FIG. 5. As can be seen in FIG. 6, each of the plurality of rungs 96 are physically isolated from each other and physically isolated from the RF shield 92. A first ground patch is physically and electrically connected to the first end of each rung and a second ground patch is physically and electrically connected to the second end of each rung. Each ground patch 98 is spaced apart from, but capacitively couples to the RF shield 92 through dielectric material 110. The RF shield 92 is the innermost layer of the inner gradient coil set 82. The RF shield 92 is spaced apart from the ground patches 98 by dielectric material 110 that substantially inhibits electrical charges from flowing therethrough, but forms a capacitor.

In an exemplary embodiment, the size of the ground patches 98 are determined to optimize the functionality of the RF body coil. Also, the type of dielectric material used and its depth may be selected and optimized to provide good capacitive coupling between the ground patches 98 and RF shield 92. The ground patches 98 provide a current return path from the rungs 96 to the RF shield 92.

Beneficially, embodiments of the invention provide an arrangement for RF cables for use in a MRI system that is configured to eliminate shield currents from the outside of the RF cable shields without the need for additional shielding and/or redesign of the resonance assembly.

This written description uses examples to disclose the subject matter of this disclosure, including the best mode, and to enable any person skilled in the art to practice the subject matter, including making and using any apparatus, devices or systems, and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A resonance assembly of a magnetic resonance imaging (MRI) system, the magnet resonance assembly comprising:
a superconducting magnet assembly;
a gradient coil assembly disposed within an inner diameter of the superconducting magnet assembly, the gradient coil assembly including a RF shield;
a RF coil assembly disposed within an inner diameter of the gradient coil assembly, the RF coil assembly comprising:
a pair of end rings with a plurality of rungs positioned between the pair of end rings;
a plurality of ground patches spaced apart from at least one of the pair of end rings; and
a plurality of RF cables, each RF cable having a plurality of conductors and a shield positioned around the plurality of conductors in a coaxial cable configuration;
wherein the shield of each of the plurality of RF cables is attached to a ground patch of the plurality of ground patches; and
wherein each of the plurality of ground patches are capacitively coupled to the RF shield.

2. The resonance assembly of claim 1, wherein the plurality of ground patches are spaced apart from the RF shield with a dielectric material in between.

3. The resonance assembly of claim 1, wherein the pair of end rings, plurality of rungs, and plurality of ground patches are applied on an outer cylindrical surface of a RF coil former.

4. The resonance assembly of claim 3, wherein the plurality of rungs are arranged cylindrically around an outer surface of the RF coil former and are uniformly spaced apart from one another.

5. The resonance assembly of claim 1, wherein the RF coil assembly is a birdcage RF body coil.

6. The resonance assembly of claim 1, wherein the RF coil assembly is a transverse electromagnetic (TEM) RF body coil.

7. The resonance assembly of claim 1, wherein the RF coil assembly is a micro-strip loop RF body coil.

8. The resonance assembly of claim 1, wherein the pair of end rings, plurality of rungs, and plurality of ground patches are a conductive foil.

9. The resonance assembly of claim 1, wherein the pair of end rings, plurality of rungs, and plurality of ground patches are a conductive tape.

10. The resonance assembly of claim 1, wherein the pair of end rings, plurality of rungs, and plurality of ground patches are a thin conductive sheet.

11. The resonance assembly of claim 1, wherein the shield of each of the plurality of RF cables is electrically connected to a ground patch of the plurality of ground patches.

12. A magnetic resonance imaging (MRI) system comprising:
a RF coil assembly;
a gradient coil assembly disposed around the RF coil assembly, the gradient coil assembly including a RF shield; and
a superconducting magnet assembly disposed around the gradient coil assembly, the superconducting magnet assembly including a vessel containing a plurality of superconducting coils;
wherein the RF coil assembly comprises:
a pair of end rings with a plurality of rungs positioned between the pair of end rings;
a plurality of ground patches spaced apart from at least one of the pair of end rings; and
a plurality of RF cables, each RF cable having a plurality of conductors and a shield positioned around the plurality of conductors in a coaxial cable configuration;
wherein the shield of each of the plurality of RF cables is attached to a ground patch of the plurality of ground patches; and
wherein the plurality of ground patches are capacitively coupled to the RF shield.

13. The MRI system of claim 12, wherein the plurality of ground patches are spaced apart from the RF shield with a dielectric material in between.

14. The MRI system of claim 12, wherein the shield of each of the plurality of RF cables is electrically connected to a ground patch of the plurality of ground patches.

15. The MRI system of claim 12, wherein the RF coil assembly is a birdcage RF body coil.

16. A method for eliminating the shield current in an RF cable of a MRI system, the method comprising:
providing a plurality of ground patches, each of the plurality of ground patches electrically coupled to a RF cable shield of a plurality of RF cables and each of the plurality of ground patches capacitively coupled to a RF shield.

17. The method of claim 17, further comprising the step of applying the plurality of ground patches on an outer cylindrical surface of a RF coil former.

18. The method of claim 16, wherein the plurality of ground patches are spaced apart from the RF shield with a dielectric material in between.
